(19) United States
(12) Patent Application Publication
(10) Pub. No.: US 2021/0033504 A1
(43) Pub. Date: Feb. 4, 2021

(54) MICRO-NANO PARTICLES DETECTION SYSTEM AND METHOD THEREOF

(71) Applicant: NATIONAL CENTER FOR NANOSCIENCE AND TECHNOLOGY, Beijing (CN)

(72) Inventor: Jiashu Sun, Beijing (CN)

(21) Appl. No.: 17/043,038

(22) PCT Filed: Aug. 3, 2018
(86) PCT No.: PCT/CN2018/098650

§ 371 Date: Sep. 29, 2020

(30) Foreign Application Priority Data
Apr. 8, 2018 (CN) 201810306599.X

(51) Int. Cl. G01N 1/40; G01N 1/44; G01N 21/64; G01N 15/06

(52) U.S. Cl. CPC G01N 1/4022; G01N 1/44; G01N 2015/0065; G01N 21/6428; G01N 15/0612; G01N 21/6402

(57) ABSTRACT

The invention relates to a micro-nano particles detection system and a method thereof. The system comprises a heating unit (1), a sample chamber unit (2), and a signal acquisition unit (4), wherein the heating unit (1) is arranged outside the sample chamber unit (2) for heating a sample in the sample chamber unit (2). Micro-nano particle fluid is loaded in the sample chamber unit (2). After the heating unit (1) heats the sample chamber unit (2), the sample chamber unit (2) generates a thermophoresis effect, so that the micro-nano particles are gathered at one side with temperature lower than the micro-nano particle fluid in the sample chamber unit (2). The signal acquisition unit (4) is used for collecting relevant information of the gathered micro-nano particles, and carrying out corresponding analysis.

### CROSS-REFERENCE TO RELATED APPLICATIONS

This application claims priority to and is the National Stage of International Application No. PCT/CN2018/098650, filed on Aug. 3, 2018, and further claims priority to CN 201810306599.X, filed on Apr. 8, 2018, the content of each of which is incorporated by reference herein.

### TECHINICAL FIELD

The invention relates to the technical field of micro-nano particle detection, in particular to a micro-nano particle detection system and method based on thermophoresis effect.

### BACKGROUND TECHNIQUE

In the prior art, the detection of micro-nano particles are measured the size, shape, concentration, activity and the like of the particles, which is widely used in hematology, immunology, molecular biology, clinical medicine and other disciplines. In the prior art, the flow particle detection method is often used to detect micro-nano particles, which is a technology for quantitative analyzing and sorting the particles in the liquid one by one. The Coulter principle adopted in the detection means that when particles suspended in the electrolyte pass through a small hole along with the electrolyte, they replace the electrolyte with the same volume, which leads to an instantaneous change in the resistance between two electrodes inside and outside the small hole in the circuit designed with constant current, resulting in potential pulses. The size and frequency of pulse signals are proportional to the size and number of particles. Sample focusing is the key technology of flow particle detection. At present, the sample solution is focused by external force. Focusing can be divided into focusing with sheath fluid and focusing without sheath fluid.

Among them, sheath fluid focusing, as disclosed in Microfluidic Particle Instrument and Manufacturing Method published in Chinese Patent 201210482142.7, sample fluid is injected from sample fluid inlet and sheath fluid is injected from sheath fluid inlet respectively by using the pressure of external injection pump, and then the sample fluid and two sheath fluids flow to sheath flow convergence area at the same time, and the convergence of sheath liquid will pack the particles in the sample liquid into a linear arrangement and flow into the detection area for detection. In this method, two sheath flows and sample liquid need driving sources, and a motor is used to control three pipes, which not only makes the equipment huge, but also increases the cost. More importantly, because the chip needs to be replaced every time, the three channels need to be reconnected with the motor every time, and the sealing problem at this joint will affect the pressure on the three channels, resulting in poor focusing effect and inaccurate test results.

Among them, focusing without sheath fluid, as disclosed in A Microfluidic Chip Structure for Flow Particle Analyzer and Its Manufacturing Method published in Chinese Patent 201310283051.5, it adopts a conical focusing structure, which is considered to have a focusing effect similar to that of the traditional sheath fluid flow system, so that the particles flow into the microchannel individually, and the microchannel binds the particles through the channel to make them pass through the detection area individually, resulting in inaccurate detection results under the detection conditions of high concentration samples.

In the above two technical solutions for detecting micro-nano particles, on the one hand, by generating potential pulses, nano particles are separated and detected by electrochemical methods to form a stream containing micro-nano particles, and the amount of samples required is extremely large. On the other hand, the flow direction and accumulation direction of micro-nano particles are defined by a driving source such as a motor and a single channel with a fixed structure. In the process of applying external force and defining the channel, the external force acts on the fluid, and the force applied to the micro-nano particles is often uncontrollable.

Especially for the detection of micro-nano biological particles, such as exosomes, which are membrane vesicles secreted by cells and used for intercellular communication. Because they contain proteins and genetic materials related to mother cells, they can regulate a variety of physiological or pathological reactions, including tumor cell invasion and metastasis, vascular growth, immune response, etc. In recent years, exosomes have gradually become a new biomarker for non-invasive tumor diagnosis. It is often necessary to analyze the surface protein types of exosomes in tumor diagnosis. However, due to the lack of accurate, feasible and easy-to-operate analysis methods, there are still challenges in analyzing the small differences of different exosomes' surface proteins.

It is commonly used in the prior art: first, enzyme linked immunosorbent assay (ELISA) refers to a qualitative and quantitative detection method which combines soluble antibodies to solid-phase carriers such as polystyrene, and makes use of antigen-antibody binding specificity to carry out immune reaction. During the determination, the tested specimen (the antibody in which is determined) and the enzyme-labeled antibody react with the antigen on the surface of the solid-phase carrier according to different steps; the antigen-antibody complex formed on the solid-phase carrier is separated from other substances by washing method, and finally the amount of enzyme bound on the solid-phase carrier is proportional to the amount of tested substances in the sample. After adding the substrate of enzyme reaction, the substrate is converted into colored product by enzyme catalysis, and the amount of the product is directly related to the amount of the tested substance in the specimen, so it can be qualitatively or quantitatively analyzed according to the depth of color reaction.

Second, Western Blot, the basic principle of western blot, is to color the cell or biological tissue samples treated by gel electrophoresis with specific antibodies; by analyzing the position and depth of staining, the information about the expression of specific proteins in the analyzed cells or tissues can be obtained.

The above two technical solutions, on the one hand, carry out complex pretreatment, separation and purification and heavy operation steps on samples, and need to adopt special equipment and methods; on the other hand, the detection method requires a large number of samples, and the process of cancer detection for serum is often not adaptable.

### SUMMARY OF THE INVENTION

The purpose of the present invention is to provide a micro-nano particle detection system and method to overcome the above technical defects.

In order to achieve the above object, the present invention provides a micro-nano particle detection system, comprising a heating unit and a sample chamber unit, wherein,

said heating unit is used to heat a sample in the sample chamber unit;

said sample chamber unit is loaded with micro-nano particle fluid, and after said heating unit heats said sample chamber unit, thermophoresis effect is generated in said sample chamber unit, so that micro-nano particles are aggregated on the side of said sample chamber unit with a temperature lower than that of the micro-nano particle fluid for detection.

Further, said system further comprises a signal collecting unit, said signal collecting unit collects related information of the aggregated micro-nano particles and performs corresponding analysis.

Further, said sample chamber unit comprises a sealed sample chamber for loading said micro-nano particle fluid and for providing a space for generating thermophoresis effect, said sample chamber comprising: a second heat conducting surface for sealing the sample chamber and accumulating the micro-nano particles, wherein the temperature near the second heat conducting surface is lower than the temperature of the micro-nano particle fluid, so that a temperature difference is generated between the second heat conducting surface and the micro-nano particle fluid, a thermophoresis effect is generated, and micro-nano particles are driven to move directionally to the second heat conducting surface.

Further, said heating unit is a laser which irradiates said sample chamber unit, and light beams pass through the micro-nano particle fluid and the second heat conducting surface in turn to generate thermophoresis effect on the micro-nano particle solution.

Further, the sample chamber further comprises: a first heat conducting surface for sealing the sample chamber, wherein the second heat conducting surface and the first heat conducting surface can both pass light beams.

Further, said second heat conducting surface is made of transparent material, which is made of sapphire or diamond; the first heat conducting surface is any one or combination of glass, polymethyl methacrylate, polydimethylsiloxane and sapphire.

Further, said micro-nano particles are exosomes, extracellular vesicles, cells or microspheres with good biocompatibility.

Further, said micro-nano particles are immune microspheres combined with target biomolecules, and the immune microspheres are prepared by fixing antibodies or aptamers on the surfaces of the microspheres.

The present invention further provides a method for detecting micro-nano particles, characterized in that, comprising: heating fluorescent-labeled micro-nano particle fluid in the sample chamber unit to generate temperature difference in the sample chamber unit so as to generate thermophoresis effect in the sample chamber unit, so as to aggregate the fluorescent-labeled micro-nano particles on the side of the sample chamber unit whose temperature is lower than that of the micro-nano particle fluid, so as to amplify labeled fluorescent signals;

step b, collecting the corresponding index information of the micro-nano particles and analyzing the corresponding indexes through the micro-nano particles aggregated at the low temperature side in the sample chamber unit.

Further, the micro-nano particles are exosomes or immune microspheres combined with target biomolecules, and the immune microspheres are prepared by fixing antibodies or aptamers on the surfaces of the microspheres.

Compared with the prior art, the micro-nano detection system of the present invention has the beneficial effects that by heating one direction of the sample chamber unit where micro-nano particles are located, thermophoresis effect and convection are introduced, so that temperature difference is generated in the sample chamber unit, and low temperature is generated on the side far away from the heating unit, and thermophoresis effect causes micro-nano particles in samples to migrate and accumulate in the sample chamber unit, so as to complete the accumulation of micro-nano particles; at the same time, convection is generated in the sample chamber unit due to buoyancy generated by thermal expansion of the sample liquid. In the low temperature area of the sample chamber unit, the direction of convection points from the periphery to the heating area of the sample chamber unit, which further promotes the accumulation of micro-nano particles. The lower surface of the sample chamber is designed as a transparent material with excellent thermal conductivity, which makes the exosomes migrate to the lower surface of the sample chamber with lower temperature. At the same time, convection is generated in the sample chamber unit due to buoyancy generated by thermal expansion of the sample liquid, which can accelerate and strengthen the aggregation of exosomes, thus improving the signal amplification factor. Further, the system incubates the sample to be tested containing exosomes with fluorescently labeled aptamers or antibodies, and the exosome is labeled with fluorescence through the specific combination of aptamers or antibodies with exosome surface protein. The incubated samples are put into the transparent sample chamber and placed on the fluorescent microscope stage for observation. The infrared laser irradiates the samples through the sample chamber, and the exosomes in the samples are highly enriched at the laser spot at the bottom of the sample chamber by thermophoresis, so that the exosomes fluorescence is highly amplified, and the abundance of a certain exosome surface protein is detected by fluorescence intensity.

Furthermore, the system uses laser to irradiate and heat the sample chamber, and transparent heat conducting surfaces with different heat conducting properties are arranged on the opposite sides of the sample chamber, so that a temperature difference is generated between the two heat conducting surfaces to generate thermophoresis effect and drive micro-nano particles to directionally move from the first heat conducting surface to the second heat conducting surface with lower temperature. Especially, the use of beam heating does not require other auxiliary equipment, as long as the transparent heat conducting surface is arranged above and below the sample chamber. In addition, the stress of micro-nano particles under thermophoresis effect is proportional to the square of particle diameter, but is independent of the number of micro-nano particles. Therefore, only a small amount of micro-nano particles can be used for aggregation and detection, and only 0.1 microliter of sample dosage is needed for exosomes. It is convenient to operate, does not need special instruments, and does not need sample pretreatment and exosomes purification, and is generally applicable to aptamers and antibodies; it is not limited to exosomes, but other extracellular vesicles, cells and other micro-nano biological particles can be used.

In particular, that micro-nano particle detection system and method of the present invention can select a specific temperature to complete the measurement without been limited by the specific temperature, and is only need to generate temperature difference to accumulate particles. It can also be measured in various solution environments, including the complex detergent environment needed to study membrane proteins. It can also detect various molecules with different sizes, such as ions, nucleic acid fragments, nucleosomes and liposomes. During the specific detection, the system can adjust the temperature difference, the height between the upper and lower heat conducting surfaces, the type of fluid and the frequency of laser irradiation according to the physical properties of the particles and the size of the particles. The adjustment of the above parameters can realize quantitative adjustment, with precise control and convenient adjustment.

According to the present invention, the biomacromolecules such as free proteins and nucleic acids or biomacromolecules such as proteins and nucleic acids which are not exposed on the surface of the exosomes are modified with antibodies or aptamers which can be specifically combined with target proteins and nucleic acids on the surface of micron-sized spheres to obtain immune microspheres, which are incubated with samples containing target biomacromolecules, combined with target biomacromolecules and labeled with fluorescence, so that free particles or target biomacromolecules which are not exposed on the surface are aggregated and detected after aggregatation.

According to the present invention, particles are accumulated based on the thermophoresis effect, and the loading container of the micro-nano particles is not limited, especially in a container with large volume, the particles are easier to accumulate under the thermophoresis effect, and carrier containers such as capillary tubes are not needed for guiding.

### BRIEF DESCRIPTION OF THE DRAWINGS

FIG. 1 is a structural block diagram of the micro-nano particle detection system of the present invention.

FIG. 2 is a structural block diagram of an exosome-based signal detection process of the present invention.

FIG. 3 is a comparative map of exosomes of Example 1 of the present invention before and after the test.

FIG. 4 is a schematic diagram of each surface protein map and the corresponding expression amount of each surface protein of exosomes in Example 2 of the present invention after detection.

FIG. 5 is a schematic diagram showing the expression levels of various proteins in serum exosomes of various cancer patients and healthy people in Example 2 of the present invention.

FIG. 6 is a schematic diagram of fluorescence measurement gray value of fluorescent polystyrene microspheres with different diameters according to Example 3 of the present invention.

FIG. 7 is a schematic diagram showing the expression levels of 11 protein markers in serum of ovarian cancer patients and healthy people in Example 4 of the present invention.

FIG. 8 is a schematic diagram showing the correct rate of 11 different markers and their sum as a standard for distinguishing cancer from health in Example 4 of the present invention.

### DETAILED DESCRIPTION OF EMBODIMENTS

The above and other technical features and advantages of the present invention will be described in more detail with reference to the accompanying drawings.

Preferred embodiments of the present invention will be described below with reference to the accompanying drawings. It should be understood by the person skilled in the art that these embodiments are only used to explain the technical principle of the present invention, and are not intended to limit the protection scope of the present invention.

It should be noted that, in the description of the present invention, the terms of the direction or position relationship indicated by the terms “upper”, “lower”, “left”, “right”, “inside” and “outside” etc. are based on the direction or position relationship shown in the drawings, which is only for convenience of description, but does not indicate or imply that the device or element must have a specific orientation, be constructed and operated in a specific orientation, so it cannot be understood as a limitation of the present invention. In addition, the terms “first” and “second” are only used for descriptive purposes, and cannot be understood as indicating or implying relative importance.

In addition, it should be noted that in the description of the present invention, unless otherwise specified and limited, the terms “installation”, “link” and “connection” should be understood in a broad sense, for example, they can be fixed connection, detachable connection or integrated connection; it can be connected mechanically or electrically; it can be directly connected, indirectly connected through an intermediate medium, or communicated inside two elements. For a person skilled in the art, the specific meanings of the above terms in the present invention can be understood according to specific conditions.

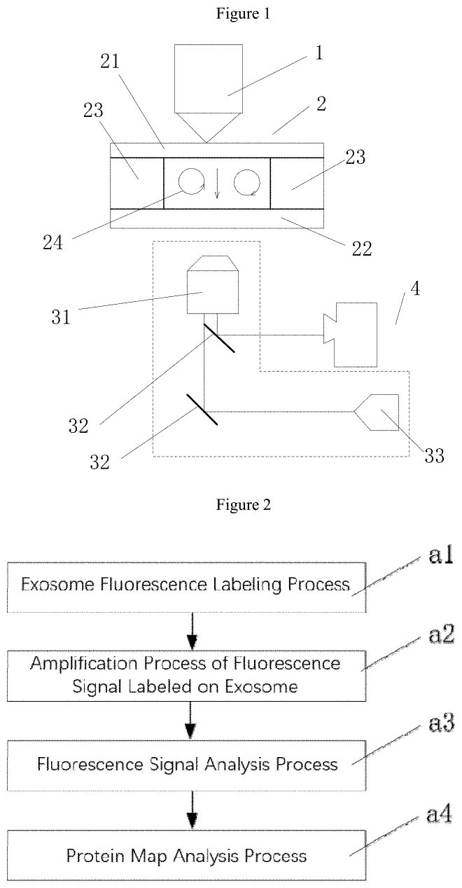

Please refer to FIG. 1, which is a structural block diagram of the micro-nano particle detection system of the present invention. The system of this example includes a heating unit 1, a sample chamber unit 2 and a signal acquisition unit 4, wherein the heating unit 1 is arranged outside the sample chamber unit 2 for heating the sample in the sample chamber unit 2. The micro-nano particles are loaded in the sample chamber unit 2, and after the heating unit 1 heats the sample chamber unit 2, thermophoresis effect is generated in the sample chamber unit 2 to aggregate the micro-nano particles on the side of the sample chamber unit 2 far away from the heating unit 1. The signal acquisition unit 4 collects the related signal information of the micro-nano particles after the micro-nano particles in the sample chamber unit 2 are accumulated, and performs corresponding analysis on the corresponding micro-nano particles. In this system, thermophoresis effect, that is the directional migration of objects under the action of temperature gradient, is used to generate a temperature gradient field locally in the sample by infrared laser irradiation, so that the exosomes in the sample migrate to the place with lower temperature. By heating one direction of the sample chamber unit 2 where micro-nano particles are located, introducing thermophoresis effect and convection, the temperature difference between micro-nano particle fluid in the sample chamber unit 2 and one side of the sample chamber unit 2 is generated, and the temperature of one side of the sample chamber unit 2 is lower than that of micro-nano particle fluid, and the thermophoresis effect causes the micro-nano particles in the sample to migrate and accumulate to the low temperature side of the sample chamber unit 2. At the same time, convection is generated in the sample chamber unit 2 due to buoyancy generated by thermal expansion of the sample fluid. In the low-temperature area of the sample chamber unit 2, the convection direction points from the periphery to the heating area of the sample chamber unit 2, as indicated by the arrow in FIG. 1, which acts as a conveyor belt to aggregate the surrounding micro-nano particles on the low-temperature side of the sample chamber unit 2, thus playing the role of aggregating micro-nano particles.

In particular, the heating unit 1 in this example is a laser, which is arranged outside the sample chamber unit 2 and irradiates the inside of the sample chamber unit 2 to generate a circular heating area inside it, although the heating area can also be linear or in other ways. A person skilled in the art can understand that the heating method is not limited to laser irradiation, and the laser irradiation direction only needs to ensure the generation of heat source. The selection of power depends on the irradiation direction, spot diameter, wavelength and other factors, and can be changed according to the actual micro-nano particles and the use environment.

In particular, the sample chamber unit 2 includes a sealed sample chamber 24 loaded with micro-nano particle samples and used to provide a space for generating thermophoresis effect. The sample chamber 24 includes a first heat conducting surface 21 for sealing the sample chamber 24 and a second heat conducting surface 22 for sealing the sample chamber 24. In this example, temperature difference is generated between the temperature of the micro-nano particle fluid loaded in the sample chamber 24 and the second heat conducting surface 22 to generate thermophoresis effect, which drives micro-nano particles from micro-nano particle fluid to the second heat conducting surface. Therefore, in this example, the temperature near the second heat conducting surface 22 is lower than the temperature of the micro-nano particle fluid.

In this example, a laser is used to heat the sample chamber 24. The first heat conducting surface 21 and the second heat conducting surface 22 are arranged opposite to each other. The second heat conducting surface 22 has higher heat conductivity than the first heat conducting surface 21, and both heat conducting surfaces are made of transparent materials, which is convenient for observing micro-nano particles. The second heat conducting surface 22 has higher heat dissipation performance than the first heat conducting surface 21. Therefore, the temperature of the second heat conducting surface 22 is lower than that of the first heat conducting surface 21. The sample chamber 24 also includes a gasket 23 for sealing the sample chamber 24. A person skilled in the art can understand that the two heat conducting surfaces 21 can be arranged opposite to each other or adjacent to each other, or arranged at a preset included angle with each other, only by driving the micro-nano particles to move and accumulate in a set direction. It can be understood by a person skilled in the art that the fluid in this example can be liquid, such as water or a mixture of water, or gas, such as heated gas or natural gas, as long as it can load micro-nano particles and allow micro-nano particles to move freely in the fluid. At the same time, the first heat conducting surface 21 and the second heat conducting surface 22 are transparent, which can pass through the first heat conducting surface and the second heat conducting surface in turn by infrared rays and bring heat into the fluid.

As a preferred example, the first heat conducting surface 21 is glass, polymethylmethacrylate (PMMA), polydimethylsiloxane (PDMS), sapphire, etc., and the second heat conducting surface 22 is sapphire or diamond with good heat conductivity. The laser irradiates the first heat conducting surface 21, the sample chamber 24 loaded with micro-nano particles and the second heat conducting surface 22 in sequence to generate a low-temperature area on the second heat conducting surface 22. The laser focus is adjusted to the sample chamber 24, and the sample liquid in the laser passing area in the sample chamber 24 absorbs the laser and the temperature rises. The thermophoresis effect causes the micro-nano particles in the sample to migrate to the second heat conducting surface 22 with lower temperature, and at the same time, convection is generated in the sample chamber unit due to buoyancy generated by thermal expansion of the sample liquid. In the low temperature direction near the second heat conducting surface 22, the convection direction points from the periphery to the laser irradiation point, which acts as a conveyor belt to aggregate the surrounding micro-nano particles in the area of the second heat-conducting surface 22 of the sample chamber below the laser irradiation point, thereby enhancing the accumulation of micro-nano particles.

In this example, the micro-nano particles are selected as exosomes, which are membrane vesicles secreted by cells and used for intercellular communication. Because they contain proteins and genetic materials related to mother cells, exosomes have gradually become a new biomarker for non-invasive tumor diagnosis in recent years.

The specific principle of this example based on exosomes is as follows.

Exosome thermophoresis model:

v_{T}=−S_{T}D∇T (1)

Wherein v_{T} is thermophoresis velocity, S_{T} is Soret coefficient, D is diffusion coefficient, ∇T is temperature gradient, and the negative sign at the right end of the model formula indicates that thermophoresis direction is low temperature direction.

The formula for calculating the Soret coefficient in the above formula (1) is:

$${S}_{T} = \frac{A}{k T} ( - {s}_{h y d} + \frac{β {σ}_{eff}^{2} {λ}_{D H}}{4 {ɛɛ}_{0} T} ) \qquad ( 2 )$$

Wherein A is exosome surface area, k is Boltzmann constant, T is temperature, S_{hyd} is hydration entropy, β is coefficient, σ_{eff} is surface equivalent charge density of the exosome, λ_{DH} is Debye length, ε₀ is vacuum dielectric constant, ε is relative dielectric constant. Based on the above formulas (1)-(2), it can be seen that the thermophoresis force of the exosomes is proportional to the square of diameter.

Migration model of exosomes in thermal convection is:

$$\begin{matrix}\frac{d {v}_{p}}{d t} = \frac{18 η}{{ρ}_{p} {a}^{2}} \frac{{C}_{D} {Re}_{s}}{24} ( u - {v}_{p} ) + \frac{g ( {ρ}_{p} - ρ )}{{ρ}_{p}} + \frac{1}{2} \frac{ρ}{{ρ}_{p}} \frac{d ( u - {v}_{p} )}{dt} & ( 3 ) \\ {C}_{D} = {a}_{1} + \frac{{a}_{2}}{{Re}_{s}} + \frac{{a}_{3}}{{Re}_{s}^{2}} & ( 4 ) \\ {Re}_{s} = ρa \vert u - {V}_{p} \vert / η & ( 5 )\end{matrix}$$

Wherein Vₚ is velocity of exosomes under the action of thermal convection, a is diameter of exosome, u is velocity of thermal convection, C_{D} is viscosity coefficient, which can be calculated according to formula (4), wherein, a₁, a₂, and a₃ are constants, Reₛ is relative motion Reynolds number, which can be calculated according to formula (5), g is gravitational acceleration, ρₚ is average density of exosomes, ρ is density of the sample liquid, η is dynamic viscosity of the sample liquid. Based on the formulas (3)-(5), it can be seen that the viscous resistance of exosome to heat convection is proportional to the diameter.

Comparing the thermophoresis force with the viscous resistance of thermal convection, it can be seen that the larger the object is, the more dominant the thermophoresis force is and the more inlined it is to aggregate at the bottom of the sample chamber. The smaller the object is, the more dominant the viscous resistance to thermal convection is, and the more inclined it is to follow the thermal convection rather than aggregate.

With continued reference to FIG. 1, the signal amplification unit 3 includes a microscope arranged in the micro-nano particle accumulation area of the sample chamber unit 2, which includes an objective lens 31 aligned with the accumulated micro-nano particles, a reflector 32 and an observation light source 33, so that micro-nano particles can be observed more clearly through the microscope. The signal acquisition unit 4 is a CCD camera. Of course, it can also be any instrument capable of detecting optical signals, taking pictures of micro-nano particles through a microscope to obtain information.

In this example, for exosome signal detection, firstly, the exosome sample is incubated with the fluorescently labeled aptamer, so that the fluorescently labeled aptamer specifically binds to the target protein on the exosome surface, thereby labeling the exosome with fluorescence. Put the incubated exosome sample into the sample chamber 24, and introduce thermophoresis effect and convection by laser heating to amplify the fluorescence signal labeled on the exosome in the sample chamber. The fluorescence signals before and after laser irradiation are recorded by CCD, and the abundance of target protein on exosome surface is obtained by analyzing the fluorescence signals before and after laser irradiation. Using a series of aptamers that can bind different target proteins, the exosome surface protein map can be obtained, and finally determine the corresponding index parameters of exosome through the analysis.

In this example, the detection method of micro-nano particles includes:

Step a, heating the micro-nano particle sample in the sample chamber unit 2 from one side to generate thermophoresis effect in the sample chamber unit 2, so as to aggregate the micro-nano particles on the low temperature side in the sample chamber unit 2;

step b, collecting the corresponding index information of the micro-nano particles and analyzing the corresponding indexes through the micro-nano particles aggregated at the low temperature side in the sample chamber unit 2.

In the above step a, convection is generated in the sample chamber unit 2 due to buoyancy generated by thermal expansion of the sample liquid. In the low temperature area of the sample chamber unit 2, the convection direction points from the periphery to the heating area of the sample chamber unit 2, and the surrounding micro-nano particles are aggregated on the low temperature side of the sample chamber unit 2.

In particular, the example performs signal detection on exosomes. As shown in FIG. 2, the process is as follows:

Step a1, the exosome sample is incubated with fluorescently labeled aptamer, so that the fluorescently labeled aptamer is specifically bound with target protein on the surface of exosome, thereby labeling the exosome with fluorescence;

Step a2, placing the incubated exosome sample into the sample chamber, introducing thermophoresis effect and convection by laser heating, and aggregating the exosome on the low temperature side of the sample chamber, so as to amplify the fluorescence signal labeled on the exosome in the sample chamber;

Step a3, obtaining fluorescence signals before and after light irradiation, and obtaining the abundance of target protein on the exosome surface by analyzing the fluorescence signals before and after laser irradiation;

Step a4, using a series of aptamers capable of binding different target proteins to obtain exosome surface protein map.

The above micro-nano particle detection system and method will be described by specific examples below.

### EXAMPLE 1

The exosome samples are incubated with fluorescent labeled aptamers, and the selected aptamers are oligonucleotide fragments which can specifically bind proteins or other small molecular substances, which are screened by in vitro screening technology SELEX (Systematic Evolution of Ligands by Exponential Enrichment). In particular, the fluorescent labeled aptamers are single-stranded DNA with 20-60 bases, and the clew diameter in the sample liquid is less than 5 nanometers, while the diameter of exosome is 30-150 nanometers. The aptamer specifically recognizing CD63 protein is applied to the exosomes in the culture supernatant of A375 cells (human melanoma cells). Fluorescent groups can be modified at the end of aptamer by standard means. When the aptamer interacts specifically with the target protein on the surface of exosome, the exosome are labeled with the fluorescence carried by aptamer. The exosome sample in this example is the supernatant of cell culture medium, and the incubation conditions of the samples are: the incubation time is 2 hours; the aptamer concentration is 0.1 micromole per liter, and the incubation temperature is room temperature.

Among them, the laser uses infrared laser with a wavelength of 1480 nm for sample heating, with a power of 200 mW and a spot diameter of the focused laser of about 200 microns. Since the main component of sample liquid is water, which has an absorption peak near the 1480 nm band. It can be understood by the person skilled in the art that the heating method is not limited to laser irradiation, and the wavelength is not limited to 1480 nm. The laser irradiation direction is not limited to top-down irradiation, and the selection of power depends on the irradiation direction, spot diameter, wavelength and other factors, not limited to 200 mW. In this example, the laser is irradiated from top to bottom, the upper heat conducting surface of the sample chamber is made of transparent materials, such as glass, PMMA and PDMS, and the lower heat conducting surface is made of sapphire with better heat conductivity, so that a low temperature area is formed on the bottom surface, so that exosome thermophoresis aggregates on the bottom surface. The thickness of the upper heat conducting surface is 1 mm, the thickness of the lower heat conducting surface is 1 mm, and height of the middle gasket and the sample chamber is 240 mm.

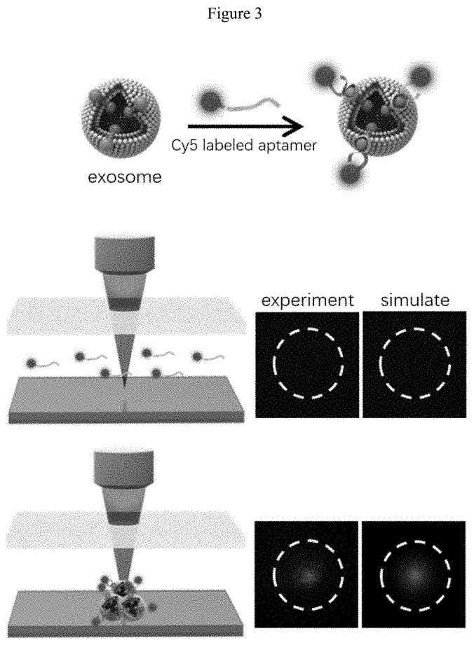

According to the above signal detection method based on exosome, when the aptamer recognizes and binds to the exosome surface protein, the fluorescent label on the aptamer follows the exosomes and is aggregated in the bottom area of the sample chamber below the laser spot, and enhanced fluorescent signal is generated. When the aptamer does not recognize the exosome surface protein, the free aptamer could not aggregate because of its small size, and the signal is not enhanced. As shown in FIG. 3, in this example, CD63 protein is widely present on the exosome surface of various cells, and obvious fluorescence signal appears after laser irradiation, indicating that the exosome surface of A375 cells has CD63 protein.

Fluorescence microscope is used to excite and receive the fluorescence signal labeled on the aptamer after binding to the exosome, and the wavelength of excitation and reception of fluorescence is related to the characteristics of the labeled fluorescent luminescent group. In this example, the excitation/emission wavelength of the luminescent group Cy5 is 649/666 nm, and the fluorescence signal is recorded by CCD connected to the fluorescence microscope. The fluorescence signals before and after laser irradiation are recorded by CCD, and the abundance of target protein on exosome surface is obtained by analyzing the fluorescence signals before and after laser irradiation.

### EXAMPLE 2

In this example, serum samples of cervical cancer patients are used, and the abundance of seven exosome surface proteins (CD63, PTK7, EpCAM, HepG2, HER2, PSA, CA125) in serum samples is detected by using seven different aptamers, and compared with serum samples of healthy people.

The exosome operation method is used, and the laser, the sample chamber, the microscope and the CCD camera are the same.

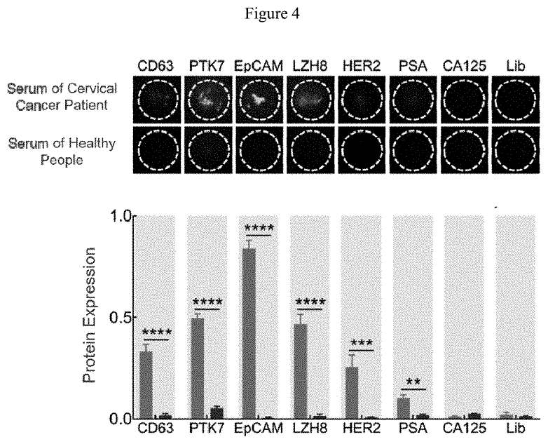

As shown in FIG. 4, it can be seen that the serum exosomes of cervical cancer patient highly express CD63 protein, and cancer-related markers PTK7, EpCAM, HepG2, HER2, PSA and CA125, among which CA125 can be used as a traditional marker of cervical cancer, and some cervical cancer patients have high expression of HER2. It is generally believed that tumor markers PTK7 and EpCAM are related to various cancers, HepG2 is specific for liver cancer, and PSA is specific for prostate cancer. However, these tumor markers are not strictly related to certain cancers. However, during the growth of tumor or the metastasis of cancer to other organs, after many times of division and proliferation, the cells constantly produce gene mutations and present changes in molecular biology or genes, so these tumor markers are not strictly related to a certain cancer. In this example, PTK7, EpCAM and HepG2 are detected in the serum of the cervical cancer patient, which showed the potential of this method in capturing gene mutation or metastasis of tumor. In addition, as a protein commonly expressed in exosomes, the expression of CD63 is higher in cancer patients than in healthy people, which is consistent with the results obtained by traditional detection methods.

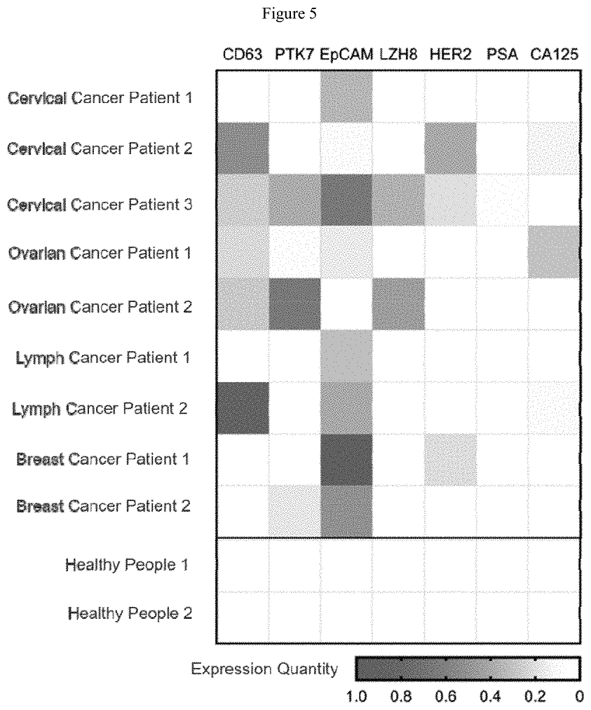

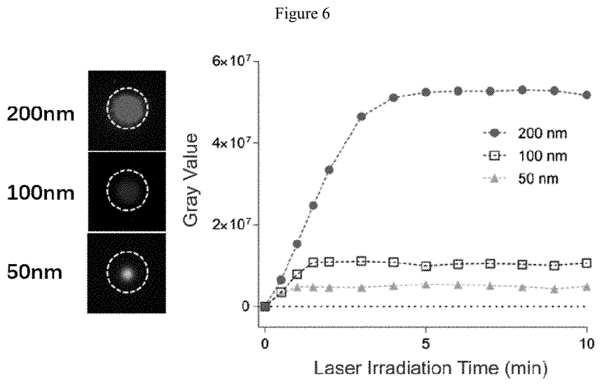

The method is further applied to a large number of real clinical serum samples, including 3 cases of cervical cancer, 2 cases of ovarian cancer, 2 cases of lymph cancer, 2 cases of breast cancer and 2 cases of healthy people. As shown in FIG. 5, this method can detect the difference of protein expression in serum exosomes between various cancer patients and healthy people. The expression of serum exosome protein is different among different kinds of cancers, which mainly shows that HER2 is highly expressed in breast cancer and cervical cancer, CA125 is highly expressed in ovarian cancer and cervical cancer, PSA is not expressed in all kinds of cancers detected, and EpCAM, PTK7 and CD63 are highly expressed in many kinds of cancers. These results are consistent with the detection results of existing methods.

It shows that this method can sensitively detect the difference in the expression of exosome surface proteins, including cancer markers, between cancer patients' serum and healthy people's serum. It also shows that exosomes as cancer tumor markers are more convenient, sensitive and effective: traditional cancer screening or physical examination has limited types of tumor markers (limited by available expensive antibodies and reagents) and low sensitivity, which leads to false negative, that is, no marker is detected by the patient. For example, in this example, CA125 expression results in venous blood test report of cervical cancer patients are within the normal range. However, the method does not require expensive antibodies, and aptamers that can specifically bind to proteins of corresponding tumor markers can be used according to detection requirements.

### EXAMPLE 3

In this example, the micro-nano particles used are non-biological micro-nano particles, specifically fluorescent polystyrene microspheres, with the brand of Thermofisher and the diameter of 50 to 200 nanometers and the mass fraction of 0.001%, which are dissolved in an aqueous solution containing 0.02% of Tween20. The laser, the sample chamber, the microscope and the CCD camera are the same as those in the above Example 1 and 2.

As shown in FIG. 5 below, all fluorescent microspheres with different diameters are highly aggregated at the laser spot, and according to the gray value of fluorescence measurement and the fluorescence picture, the aggregation degree and the fluorescence intensity increase with the increase of particle diameter, which is consistent with the working principle of this example, that is, large particles tend to aggregate. This example shows that both biological and non-biological micro-nano particles are applicable to the concept of this technical solution.

### EXAMPLE 4

In this example, the micro-nano particles are free proteins, nucleic acids and other biological macromolecules or proteins, nucleic acids and other biological macromolecules that are not exposed on the surface of exosomes. The thermophoresis effect of the above examples can not directly accumulate free biological macromolecules. Therefore, the mechanism of this example consists in modifying antibodies or aptamers that can specifically bind to target proteins and nucleic acids on the surface of micron-sized spheres to obtain immune microspheres, which are incubated with samples containing target biological macromolecules, bound with target biological macromolecules and labeled with fluorescence. The microspheres are highly aggregated by the thermophoresis, so that the fluorescence signal of the target biomacromolecule is highly amplified, and its abundance is detected by the fluorescence intensity.

The particle detection method based on the microsphere carrier in this embodiment includes:

Step a11, preparing immune microspheres, incubating the microspheres with antibodies or aptamers, and fixing the antibodies or aptamers on the surfaces of the microspheres to obtain immune microspheres. In the process, redundant antibodies or aptamers which are not bound to microspheres are washed away. In this example, the microspheres are polystyrene microspheres.

Step b11, incubating the immune microsphere with the sample to be detected, and specifically binding the target protein or nucleic acid in the sample to be detected to the antibody or aptamer on the immune microsphere so as to be fixed on the immune microsphere.

Step c11, combining the immune microspheres bound with target biomolecules prepared in the step b11 with antibodies or aptamers carrying fluorescent groups, and labeling the target biomolecules on the immune microspheres with fluorescence through specific recognition.

Step d11, heating the immune microsphere samples bound with target biomolecules in the sample chamber unit 2 from one side to generate thermophoresis effect in the sample chamber unit 2, so as to aggregate the immune microspheres bound with target biomolecules on the low-temperature side in the sample chamber unit 2, and amplifying the signal due to fluorescence label enrichment. In this process, by generating thermophoresis, the target biomolecules are captured by immune microspheres, so that the equivalent size becomes larger, and the target biomolecules is highly enriched and the signals are amplified, while non-target biomolecules are in free state, and the equivalent size is very small, so the signal can not be amplified.

Step e11, the corresponding index information of immune microspheres bound with target biomolecules is collected and analyzed by collecting the immune microspheres bound with target biomolecules aggregated at the low temperature side in the sample chamber unit 2. In this process, the fluorescence signals before and after light irradiation are obtained, and the abundance of target protein on exosome surface is obtained by analyzing the fluorescence signals before and after laser irradiation. Using a series of aptamers that can bind different target proteins, the exosome surface protein map can be obtained.

In this example, immune microspheres coated with antibodies are used to capture free protein markers in the whole blood of ovarian cancer patients, and infrared laser generated thermophoresis is used to amplify the fluorescence signals of protein markers and determine the abundance of protein markers to be detected. The results are consistent with those of traditional detection methods, which provide molecular information for cancer detection. In this example, EpCAM, CA-125, CA19-9, CD24, HER2, MUC18, EGFR, CLDN3, CD45, CD41 and D2-40 are selected as protein markers for ovarian cancer, and specific antibodies (purchased from abcam company) corresponding to these protein markers are respectively prepared into immune microspheres, and each antibody is independently prepared into microspheres specifically for the detection of a marker. There is a standard process for the preparation of antibody-coated immune microspheres, which is briefly described here: polystyrene microspheres with diameter of 1 micron are incubated with antibodies with concentration of 5 μg/ml for 1 hour at room temperature, and then surplus unreacted antibodies are removed by ultrafiltration after incubation. The diameter of microspheres is not limited to 1 micron, as long as the size reaches thermophoresis and can aggregate. The material is not limited to polystyrene, and any material can be used as long as it can successfully attach the antibody and does not affect the activity of the antibody and the protein marker to be detected. The antibody concentration and incubation temperature and time are not limited to the specific values described in this example, which can be varied with reference to the actually used antibody brand, batch and specific experimental conditions.

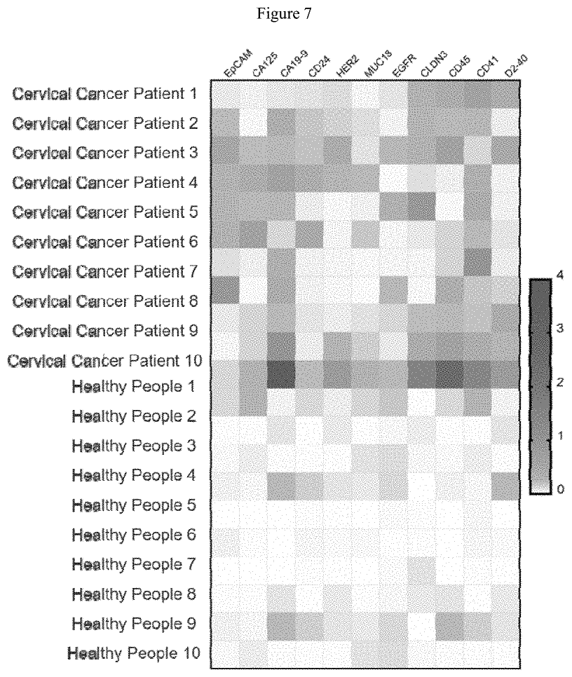

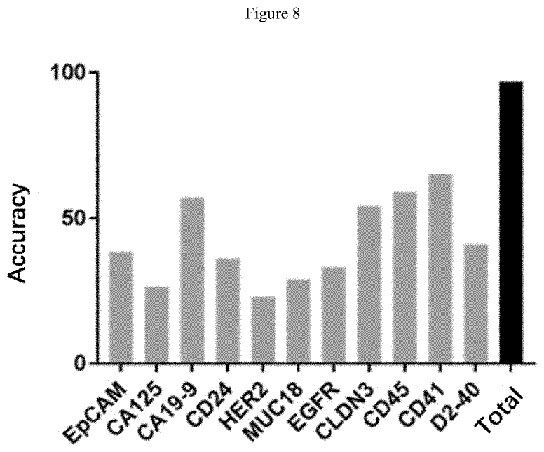

In this example, 11 kinds of immune microspheres are prepared by the above steps to detect the above 11 kinds of markers respectively. After diluting 1.1 μM of patient's serum by 100 times, they are evenly divided into 11 parts, which are respectively mixed with 11 kinds of immune microspheres and incubated for 1 hour at room temperature. The antibody with fluorescence label is incubated with the microspheres capturing the protein markers to be detected, and the protein markers are fluorescently labeled. And the detection system of each example is used for detection. The above steps are repeated for 10 ovarian cancer patients and 10 healthy people, and the expression levels of 11 protein markers in 20 serum samples are measured, as shown in FIG. 7 and FIG. 8. Due to the heterogeneity of cancer, the expression of serum markers in each patient is not exactly the same, but the overall expression level is significantly higher than that in healthy samples. It is important that each protein marker as a single cancer detection standard has low accuracy. Using the sum of protein expression in 11 as the detection standard, the detection can accurately distinguish ovarian cancer from healthy samples. Using more diagnostic markers will greatly improve the diagnostic accuracy, but the cost will increase with the increase of the number of markers, especially the antibodies against certain markers are rare and expensive. According to the detection method of this example, each marker only needs 1 ng of antibody per person, so the cost is less than that of 1 yuan, and other expensive reagents are not needed.

In the detection method of this embodiment, each marker needs only 1 ng of antibody per person, and the cost is less than 1 yuan, and no other expensive reagent is needed.

Heretofore, the technical solution of the present invention has been described with reference to the preferred embodiments shown in the drawings, but it is easy for the person skilled in the art to understand that the protection scope of the present invention is obviously not limited to these specific embodiments. On the premise of not deviating from the principle of the present invention, a person skilled in the art can make equivalent changes or substitutions to relevant technical features, and the technical solutions after these changes or substitutions will fall within the protection scope of the present invention.

1. A micro-nano particle detection system, characterized in that, comprising a heating unit and a sample chamber unit, wherein,
said heating unit is used to heat a sample in the sample chamber unit;
said sample chamber unit is loaded with micro-nano particle fluid, and after said heating unit heats said sample chamber unit, thermophoresis effect is generated in said sample chamber unit, so that micro-nano particles are aggregated on the side of said sample chamber unit with a temperature lower than that of the micro-nano particle fluid for detection.

2. The micro-nano particle detection system according to claim 1, characterized in that, said system further comprises a signal collecting unit, said signal collecting unit collects related information of the aggregated micro-nano particles and performs corresponding analysis.

3. The micro-nano particle detection system according to claim 1, characterized in that, said sample chamber unit comprises a sealed sample chamber for loading said micro-nano particle fluid and for providing a space for generating thermophoresis effect, said sample chamber comprising: a second heat conducting surface for sealing the sample chamber and accumulating the micro-nano particles, wherein the temperature near the second heat conducting surface is lower than the temperature of the micro-nano particle fluid, so that a temperature difference is generated between the second heat conducting surface and the micro-nano particle fluid, a thermophoresis effect is generated, and micro-nano particles are driven to move directionally to the second heat conducting surface.

4. The micro-nano particle detection system according to claim 3, characterized in that, said heating unit is a laser which irradiates said sample chamber unit, and light beams pass through the micro-nano particle fluid and the second heat conducting surface in turn to generate thermophoresis effect on the micro-nano particle solution.

5. The micro-nano particle detection system according to claim 4, characterized in that, the sample chamber further comprises: a first heat conducting surface for sealing the sample chamber, wherein the second heat conducting surface and the first heat conducting surface can both pass light beams.

6. The micro-nano particle detection system according to claim 5, characterized in that, said second heat conducting surface is made of transparent material, which is made of sapphire or diamond;
the first heat conducting surface is any one or combination of glass, polymethyl methacrylate, polydimethylsiloxane and sapphire.

7. The micro-nano particle detection system according to claim 1, characterized in that, said micro-nano particles are exosomes, extracellular vesicles, cells or microspheres with good biocompatibility.

8. The micro-nano particle detection system according to claim 1, characterized in that, said micro-nano particles are immune microspheres combined with target biomolecules, and the immune microspheres are prepared by fixing antibodies or aptamers on the surfaces of the microspheres.

9. A method for detecting micro-nano particles, characterized in that, comprising:
heating fluorescent-labeled micro-nano particle fluid in the sample chamber unit to generate temperature difference in the sample chamber unit so as to generate thermophoresis effect in the sample chamber unit, so as to aggregate the fluorescent-labeled micro-nano particles on the side of the sample chamber unit whose temperature is lower than that of the micro-nano particle fluid, so as to amplify labeled fluorescent signals;
step b, collecting the corresponding index information of the micro-nano particles and analyzing the corresponding indexes through the micro-nano particles aggregated at the low temperature side in the sample chamber unit.

10. The method for detecting micro-nano particles according to claim 1, characterized in that, the micro-nano particles are exosomes or immune microspheres combined with target biomolecules, and the immune microspheres are prepared by fixing antibodies or aptamers on the surfaces of the microspheres.
